(19) United States
(12) Patent Application Publication
(10) Pub. No.: US 2015/0011923 A1
(43) Pub. Date: Jan. 8, 2015

(54) Ortho Shoulder Ball

(71) Applicant: Maxwell Opoku Agyemang, South Miami, FL (US)

(72) Inventor: Maxwell Opoku Agyemang, South Miami, FL (US)

(21) Appl. No.: 13/936,740

(22) Filed: Jul. 8, 2013

(51) Int. Cl. A61F 5/01

(52) U.S. Cl. CPC A61F 5/013
USPC 602/20

(57) ABSTRACT

Shoulder pain is a common problem encountered in many medical practices. There is a protean source of such pain including adhesive capsulities, rotator cuff injuries, fractures, muscle sprains and strains. In all of these; pain is worsened by any weight-bearing on the affected shoulder during the acute or recovery phase of the injury. In some cases, shoulder pain results exclusively from frequent sleeping on one shoulder.

Hence, the Ortho Shoulder Ball which as described is a tennis size ball attached to a Velcro strap and worn around the shoulder at night to prevent one from bearing weight on the affected shoulder while sleeping.

The ball is solid but light-weight 6.5 cm and 60 g affixed to the Velcro strap and all materials skin-friendly and do not present any health hazards to the strap area except in case of possible allergic reactions.

The Ortho Shoulder Ball will be able to prevent shoulder pain by preventing weight bearing when affected patients are sleeping at night; which is not uncommon.

### SUMMARY OF INVENTION

ORTHO SHOULDER BALL is an invention that is worn at the shoulder during sleep or at bed time in people who have sustained shoulder injury and with shoulder pain.

### BACKGROUND OF INVENTION

Various shoulder injuries and shoulder pathologies present with pain during the acute or recovery phase. This pain is worsened by body weight at night when one sleeps on the affected shoulder. More so people sustain shoulder pain exclusively by sleeping on their shoulder; a common problem encountered by doctors in everyday practice. The ORTHO SHOULDER BALL is worn around the affected shoulder to prevent weight-bearing and subsequent pain.

### DESCRIPTION OF VIEWS

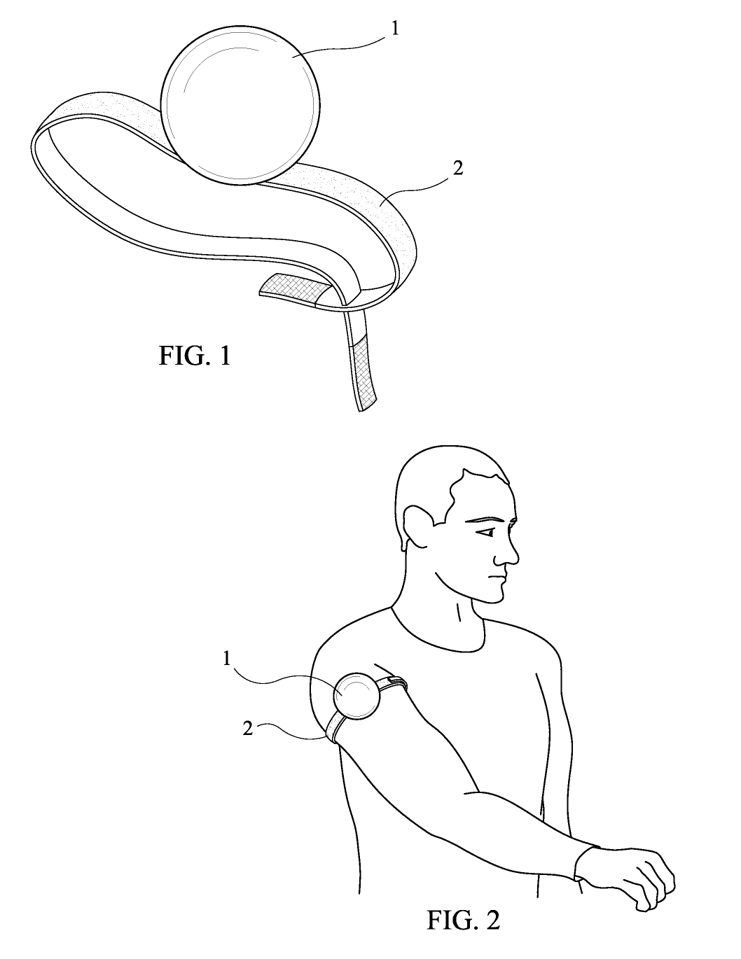

FIG. 1 shows label 1 a tennis-size ball 6.5 cm in diameter and approximately 60 g in weight affixed to label 2 a Velcro strap that can be safely fastened around the upper arm juxtaposition to the affected shoulder as shown in FIG. 1 and in such manner prevents one from rolling onto the shoulder during sleep. Materials are all skin-friendly and do not present any health hazards to the strap area except in case of allergic reactions.

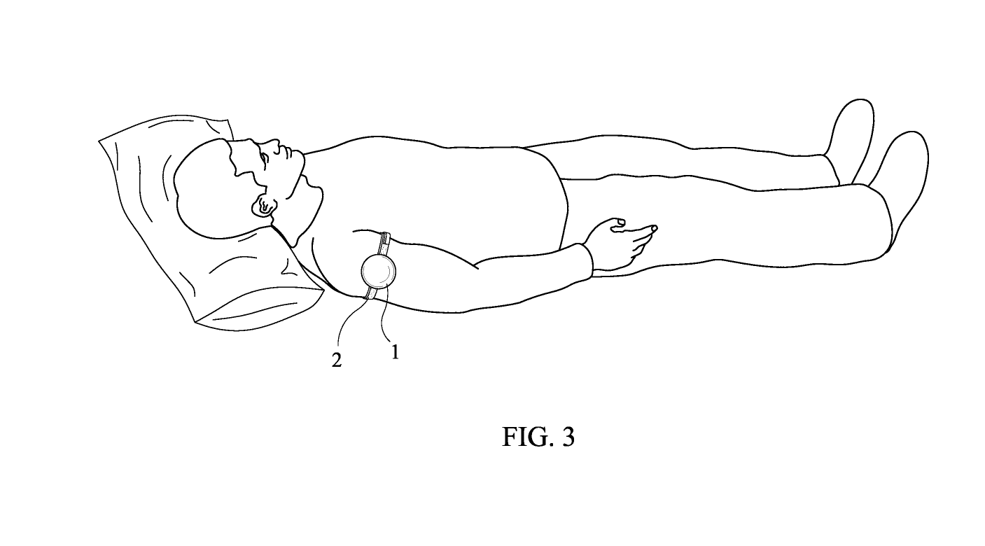

FIG. 2 shows a person wearing the Ortho Shoulder Ball.

FIG. 3 is the sleeping position with the Ortho Shoulder Ball worn during sleeping to avoid any weight bearing on the affected shoulder.

1. Shoulder pain can be primary resulting from frequent shoulder weight bearing at night and the Ortho Shoulder Ball in this invention is designed and worn over the shoulder to prevent such pain.

2. Shoulder if already injured, focal pain be secondary or worsened from sleeping on the affected shoulder hence the Ortho Shoulder Ball as depicted in this invention can be worn to avert such occurrences.
